(19) United States
(12) Patent Application Publication
(10) Pub. No.: US 2022/0152300 A1
(43) Pub. Date: May 19, 2022

(54) INTRAVENOUS INFUSION FLOW RATE REGULATION AND MONITORING

(71) Applicants: University of Washington, Seattle, WA (US); Seattle Children's Hospital d/b/a/ Seattle Children's Research Institute, Seattle, WA (US)

(72) Inventors: Yeonjin Cho, Seattle, WA (US); Joshua Daeho Chong, Seattle, WA (US); Trixie Chui-Yee Ip, Seattle, WA (US); Jan Gabrielle Silva, Seattle, WA (US); Gregory Charles Valentine, Seattle, WA (US); Krystle Perez, Seattle, WA (US); Keyi Zhou, Seattle, WA (US)

(73) Assignees: University of Washington, Seattle, WA (US); Seattle Children's Hospital d/b/a/ Seattle Children's Research Institute, Seattle, WA (US)

(21) Appl. No.: 17/529,146

(22) Filed: Nov. 17, 2021

Related U.S. Application Data
(60) Provisional application No. 63/115,771, filed on Nov. 19, 2020.

(51) Int. Cl. A61M 5/168; A61M 5/14

(52) U.S. Cl. CPC A61M 5/1689; A61M 5/1415; A61M 5/16813; A61M 2205/3306; A61M 5/16895; A61M 2205/587; A61M 5/16881

(57) ABSTRACT

An IV system can include a first flow regulator configured to restrict flow through a tube and a second flow regulator configured to restrict flow through the tube downstream of the first flow regulator. The first flow regulator can compress the tube a fixed amount to set an initial flow rate through the tube, while the second flow regulator includes a user-adjustable mechanical compression device that compresses the tube to a variable degree to fine-tune the flow rate through to a more precise flow rate that is lower than the initial flow rate. The flow regulators can utilize a purely mechanical means such that electricity is not required. Such systems can provide a simple, low-cost, and low-power way to provide precise flow control to patients, especially neonatal patient, in any environment. Systems can also include flow rate monitoring, alerting, and shutoff components.

### CROSS-REFERENCE TO RELATED APPLICATIONS

This application claims the benefit of U.S. Provisional Patent Application No. 63/115,771, filed Nov. 19, 2020, which is herein incorporated by reference in its entirety.

### FIELD

The disclosure relates to device and methods for regulating and monitoring the flow rate of intravenous infusion for patients, especially neonatal patients.

### BACKGROUND

A newborn is considered premature if it is born before the start of the 37th week of pregnancy. After birth the first 28 days of life are the most critical period in determining a preterm baby's survival rate. The highest cause of death for children under age five is prematurity. While there can be many complications associated with preterm births, neonatal dehydration is one of the top issues causing this high mortality rate. This medical condition is very common but can be preventable and treatable with appropriate equipment.

The issue of neonatal dehydration is more prevalent in low- and middle-income countries (LMIC) where healthcare is less accessible. High income countries have mitigated the problem for years with costly equipment, but such treatment has not been globalized for low-resource settings. The current standard of care is intravenous therapy (IV). However, there are no low-cost solutions to accurately deliver IV fluids at low infusion rates. Market available low-cost pumps do not have the precision required to modulate flow rates within acceptable parameters for a neonate. The lowest rate possible with existing low-cost pumps is 3 mL/h, while neonates require infusion rates as low as 1 mL/h. In terms of hydration treatment quality, there are other high-cost solutions available that have, for example, a desirable minimum delivery rate error, but the high cost makes these an unlikely option for low-resource settings.

Secondly, equipment with the capacity to monitor and control flow to this precision typically requires consistent high power energy sources, a luxury that most low resource hospitals do not have. The aforementioned solutions both require a dependable power source and are costly, thus fail to fulfill the situational requirements of being power-independent and low-cost.

Flow control options that do not use electronics, such as a roller clamp, do not have the option to precisely control infusion rates. Furthermore, these devices are designed towards adult patients and are not easily controlled at low flow rates needed for premature babies. In one exemplary low cost and completely mechanical solution, the flow rate can be controlled by substituting various tube sizes that are compatible with different device types. The sizes and flow rates available are limited in what is offered and is specific to the particular device. This is not ideal for low-resource settings that largely depend on donated products that may not have compatible individual pieces like varied sizes of tubing sets.

For electronic flow rate monitoring, there exist acceptable solutions in terms of strictly monitoring the flow rate. However, limitations of such solutions include that they do not have a feedback system to control the on/off state of the device. It is desirable for the monitor to shut off the IV infusion when the rate deviates from a rate preset by a clinician.

### SUMMARY

The herein disclosed technology can address the unmet need for resource-conscious IV treatment for dehydrated neonatal patients, though this technology can also be used for many other purposes and applications and is not limited to use with neonatal patients or otherwise. One implementation of the technology disclosed herein is sometimes referred to as “H₂neO” because of the exemplary use with neonatal patients, but using that term does not limit the technology to use with neonatal patients. This technology can provide a low-cost, external device for regulating and/or monitoring the flow rate of a gravity-operated IV infusion device. In some embodiments, the disclosed technology can integrate at least two independent components: a mechanical regulator and a monitor. The mechanical regulator can comprise gears, a mechanically operated screw, and/or other mechanical components to pinch IV tubing at a very carefully controlled degree, controlling the flow rate through the IV tube to a precise known value. The flow rate regulator components can be purely mechanical, eliminating power usage for flow rate control, while in some embodiments the flow rate regulator can include one or more non-mechanical components. The mechanical regulator can be separated into at least two parts: an initial rate setter and a variable flow regulator mechanism. The initial rate setter can apply the initial pressure onto the tube to lower the flow rate to a manageable rate. The variable flow regulator can fine tune the flow to the required rate.

The monitoring components of the system can comprise an electrical/optical monitor or other type of monitor. In some embodiments, the monitor can comprise a 3-part, semi-open-loop system, including (1) a user interface for set flow rate input, (2) a flow rate monitor, and (3) an alarm with auto shut-off of flow, which can signal a deviation of the flow rate from the user-specified flow rate. An alarm for low battery status can also be included.

An exemplary use of there disclosed system can be as follows: The user (e.g. a clinician) can use the initial rate setter block to lower the natural flow rate first (e.g. to 25 ml/hr), then the user can then input the preset flow rate on the monitoring component to specify the reference flow rate. The user can then manually fine tune the variable flow rate regulator while cross checking with the monitor to achieve the flow rate desired. The monitor can raise a flag if, for example, the detected flow rate is deviating significantly from the desired rate, physically shutting off the IV line and stopping infusion to the patient. If this happens, the user can reset the alarm on the monitor and can manually open the line to restart the system.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is an exemplary system for regulating intravenous fluid delivery to a patient.

FIG. 2 is a perspective view of an exemplary flow rate regulator, including an initial rate setter and a variable flow regulator.

FIG. 3 is an exploded view of the device of FIG. 2.

FIG. 4 is a rear view of the device of FIG. 2.

FIG. 5 is a top view of the device of FIG. 2.

FIG. 6 is an inflow side view of the device of FIG. 2.

FIG. 7 is a perspective view of the device of FIG. 2 with flow regulator raised above the flow tube.

FIG. 8 is a perspective view of the device of FIG. 2 with the flow regulator lowered to squeeze the flow tube.

FIG. 9 is an outflow side view of the device of FIG. 2, with the flow regulator lowered to squeeze the flow tube.

FIG. 10 is an outflow side view of the device of FIG. 2 with the flow regulator raised above the flow tube.

FIG. 11 is a perspective view of another exemplary flow rate regulator.

FIG. 12 shows an initial rate setter device.

FIG. 13 is a system architecture block diagram for an electronic flow monitor.

FIGS. 14A-14C illustrate a light-based flow rate monitor.

FIGS. 15A-15B illustrate a torsion spring based flow shutoff system.

FIG. 16. illustrates a solenoid-based flow shutoff system.

FIG. 17 illustrates a spring-based flow shutoff system.

FIGS. 18A-18C illustrate a clothespin-type flow shutoff system.

FIG. 19 illustrates a clamp-style shutoff system.

FIG. 20 illustrates a twisting base shutoff system.

FIG. 21 shows an exemplary flow rate monitoring chamber with a window.

FIG. 22 shows an exemplary collar for holding monitoring components.

FIGS. 23-24 illustrate an exemplary flow rate monitoring system.

### DETAILED DESCRIPTION

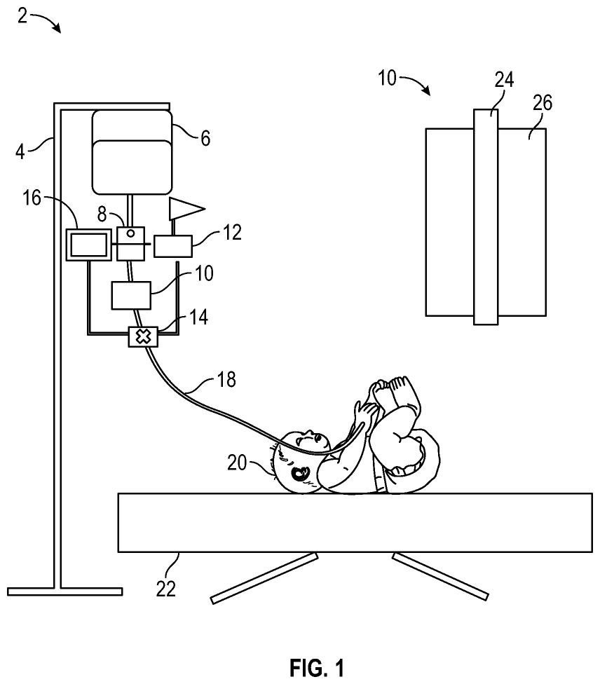

FIG. 1 illustrates an exemplary system 2 that includes a support stand 4 with a hanging IV bag 6 for providing fluids via a tube 18 to a patient 20 (e.g., a neonatal patient). The system 2 can also include a drip chamber 8, a mechanical flow-restricting device 10, and a shutoff valve 14 coupled to the tube 18 between a hanging IV bag and the patient. The system can also include additional components, such as a flow rate monitoring system 16, which can include electrical and/or optical components for sensing the drips of fluid within the drip chamber 8, and an alert or indicator 12 that signals to a user information based on the sensed flow rate in the tube. The flow-restricting device 10 (shown in detail in the inset of FIG. 1) can utilize mechanical means, such as clamping feature 26, to compress the tube 24 to restrict flow, and can be free of electronic components, which can allow it to meet desired criteria for effective flow regulation and low power use. The monitoring, alert, and shutoff components (e.g., 8, 12, 14, 16) can comprise electrical, optical, and/or mechanical components that operate at low power and can be independent of the flow regulator 10. These qualities can make the system 2 cost feasible for Low and Low Middle Income Countries (LMIC), among other cost-sensitive settings, while reducing additional health risks. Clinical staff can simply adjust the flow regulator device to pre-set flow increments and ensure the IV is connected properly to the patient. Device settings can be intuitive and require little training.

In some examples the flow rate regulator device can include an initial rate setter block that can significantly lower the flow rate, and a variable regulator device that compresses the tubing further in an extremely accurate manner. For example, one turn of a dial on the variable regulator device can correspond to 1 mm in vertical movement of a compressor platform to squeezes the flow tube. The two devices can work in series, with the initial rate setter first reducing the natural flow rate of the system to a predetermined rate, such as 25 mL/h, simply by constricting the tube a set amount, then the variable flow rate regulator device can provide the clinician with the precise control required to set the flow rate to the desired value and to make fine adjustments over time. The clinician can check the monitor for the current flow rate and make adjustments to achieve the desired flow rate.

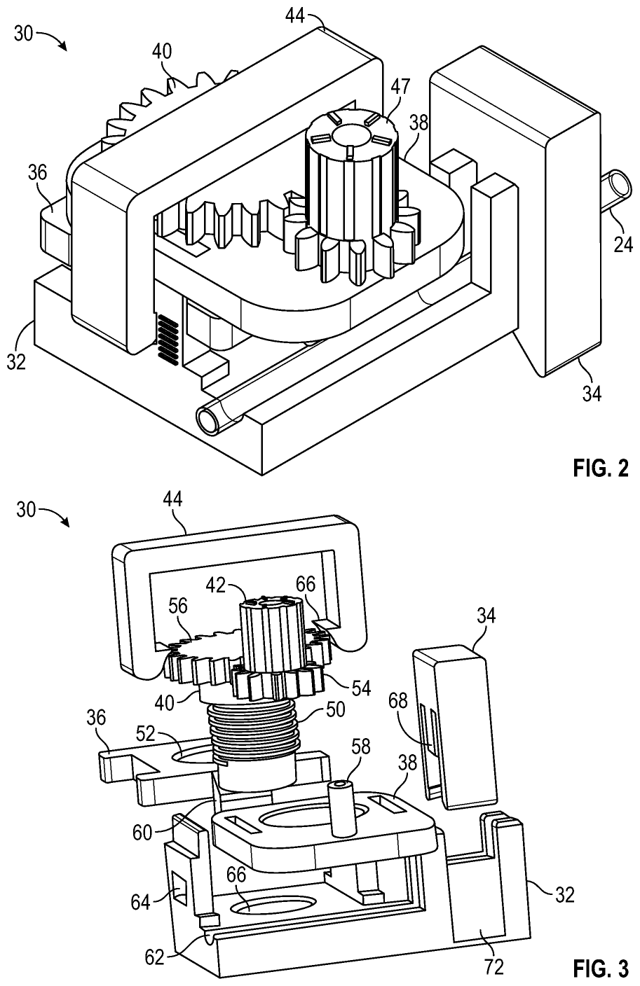

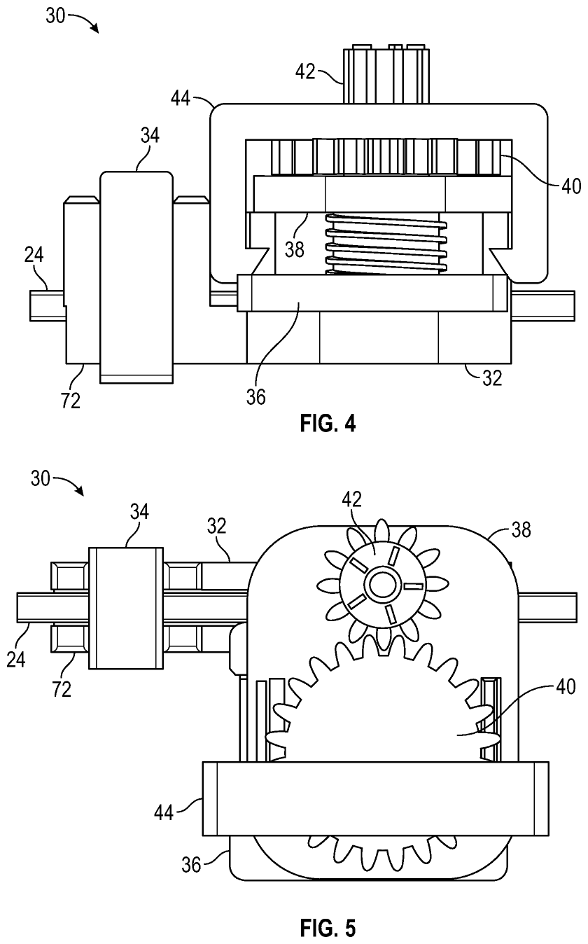

FIGS. 2-10 illustrate an exemplary flow rate regulator device 30 that includes an initial rate setter portion and a variable flow regulator portion. The device 30 can comprise a base 32 having a trough 62 that receives a flow tube 24 extending through the device 30. An initial flow rate is set by an upstream flow rate setter 34, and a secondary adjustment to the flow rate can be controlled by a variable clamping mechanism that includes a compressor platform 36 that is driven up and down relative to the base 32 by a rotating screw shaft 40, which is in turn driven by a gear coupling turned by a user. A user can rotate a dial 47 that has gear teeth 54 engaged with gear teeth 56 of the screw shaft 40. External threads 50 of the screw shaft 40 are engaged with internal threads 52 of the compressor platform 36, such that rotation of the screw shaft via rotation of the dial moves the compressor platform up and down relative to the base. A lower end of the screw shaft 40 can extend in to an opening 66 in the base. A bracket 38 is fixedly coupled to the top of base 32 and includes a stationary pin 58 about which the dial 42 rotates and an opening through which the screw shaft 40 extends and which constrains the screw shaft from translational motion while allowing the screw shaft to rotate. A retainer 44 extends around the screw shaft and compressor platform and has feet 66 that engage with openings 64 in the base 32 to fix the position of the retainer relative to the base. The retainer 44 and bracket 38 together restrict axial motion of the screw shaft 40 while allowing the screw shaft to rotate, and allowing the compressor platform 36 to move up and down between the bracket and the base. The compressor platform 36 can have lateral arms that engage with the base to restrict the compressor platform to only being allowed to move up and down relative to the base.

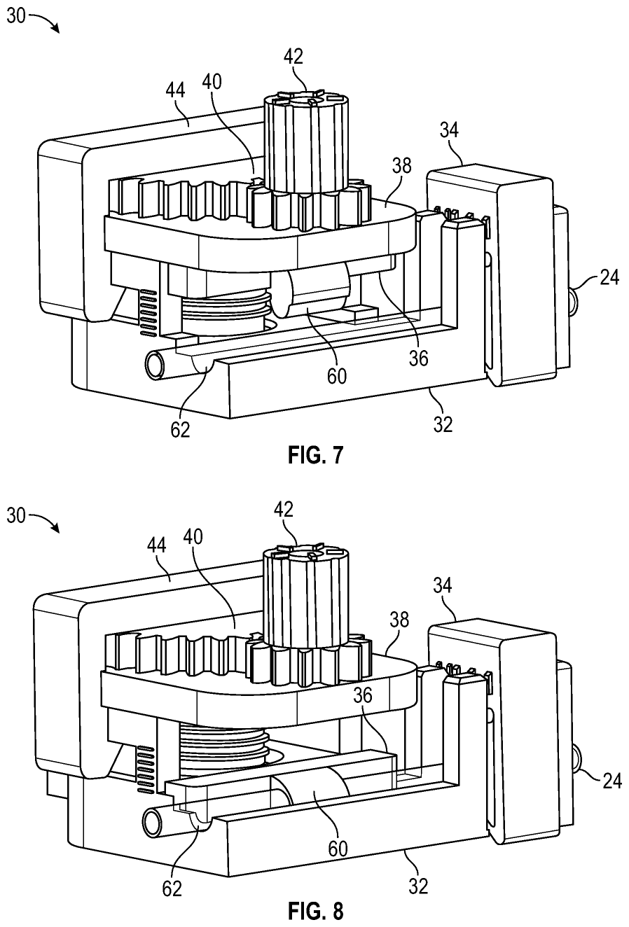

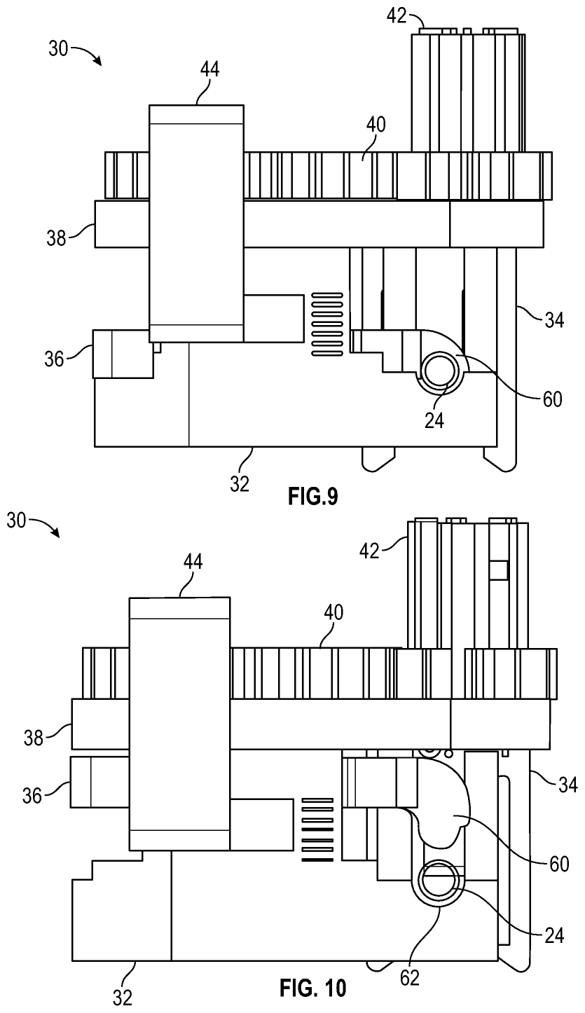

As shown in FIGS. 7-10, the compressor platform 36 includes a projection 60 that engages with the flow tube 24 to compress the tube to a variable degree in the trough 62. FIGS. 7 and 10 show an extreme uncompressed position where the projection 60 is raised above the tube 24 and is not squeezing the tube at all, while FIGS. 8 and 9 show an extreme compressed position where the projection is maximally squeezing the tube. Any intermediate position between these extremes can be achieved by adjusting the dial 42 to establish a desired degree of flow regulation through the tube. The device 30 can also include indicia (e.g., gradation markings) that assist a user to determine the exact vertical position of the projection 60 and/or how much the tube 24 is compressed.

The configuration of the device 30 can utilize mechanical advantage and surface area to significantly increase the device's precision and ease of use. The train of gears with a dial handle 42 and the threaded screw shaft 40 driving fine motion of the platform 36 add more precision to the flow rate adjustment and provide fine-tunable analog adjustability via gradual rotation of the dial by the user. In some examples, the dial 42 can half as many teeth as the screw shaft 40, providing a 2:1 gear down effect, such that two rotations of the dial are required to cause one rotation of the screw shaft. The pitch of the threads of the screw shaft and platform can also be selected to control the relationship between how much the platform moves linearly for each rotation of the dial.

This design can be functional, simple, precise, and easily manufacturable. The device 30 also does not rely on electricity, being purely mechanical, and so can be trusted to function properly at any time and location, and for any duration, without needing a power supply, recharging, new batteries, etc.

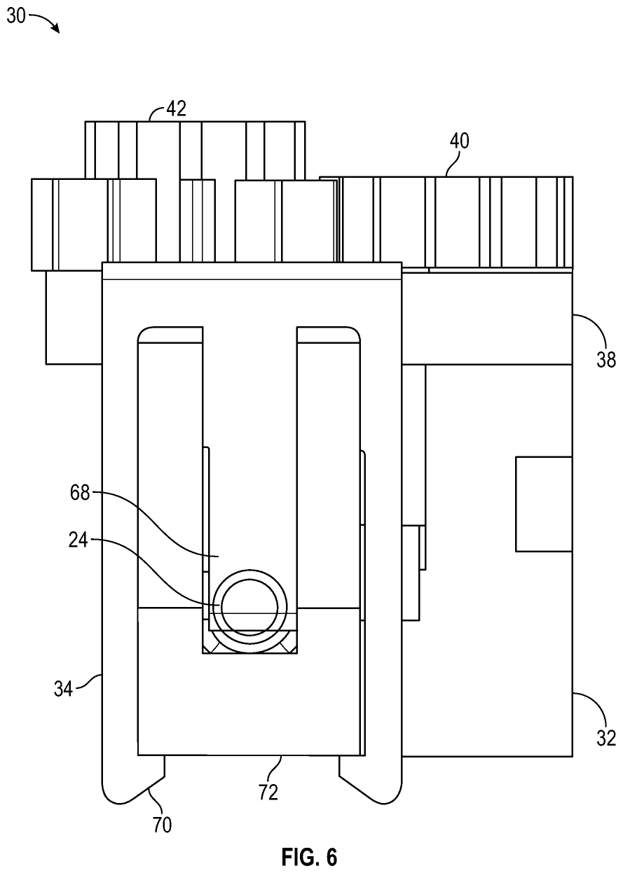

At an upstream end of the flow tube 24, an initial rate setter 34 is coupled to a receiver 72 of the base 32 with the tube squeezed between a projection 68 of the rate setter 34 and the trough 62 and sidewalls of the receiver 72 (see FIG. 6). Legs 70 of the rate setter 34 can snap around the underside of the receiver 72 to hold the rate setter in a fixed position, with a predetermined spacing between the end of the projection 68 and the trough 62 of the base. The projection structure 68 can be tightly set in between the two sidewalls of the receiver 72 to constrict the tube 24 and prevent variance to the set flow rate. A degree of incline on the outer walls of the receiver 72 can be provided to ease the process of sliding the initial rate setter 34 down over the receiver.

Different rate setters 34 with different length projections 68 can included in a set, and a user can select a desired one of the rate setters to attach to the receiver 72 to establish a desired amount of initial restriction on the tube 24, thereby setting an initial flow rate through the tube. For each rate setter in the set, the resulting gap size (e.g., distance from the end of the projection to the bottom of the trough) can be marked on the projection or elsewhere on the rate setter, for example. The set can include rate setters of sized such as 0.5 mm, 0.6 mm, 0.7 mm, 0.8 mm, 0.9 mm, 1.0 mm, 2.0 mm, etc. Or the set can include rate setters marked based on the resultant flow rate when using a given size tube, such a 0.5 mL/hr, 1 mL/hr, 2 ml/hr, 3 mL/hr, 5 mL/hr, 10 mL/hr, 15 mL/hr, etc.

In some embodiments, the device 30 can be fully 3D printable and easily manufacturable. The components of the device 30 can be manufactured using methods such as injection molding, 3D printing, casting, and/or other available methods. Furthermore, any materials can be used that are suitably rigid and durable, such as metals, polymers, composite materials, ceramics, and/or other materials. Similarly, the device can be simply and quickly assembled without additional fasteners or tools being required. For example, the parts can all snap together and hold each other in place. The device 30 can therefore be precise and functional for flow rate setting, low cost, easy to manufacture and assemble, reliable and functional in any environment, and have a compact configuration.

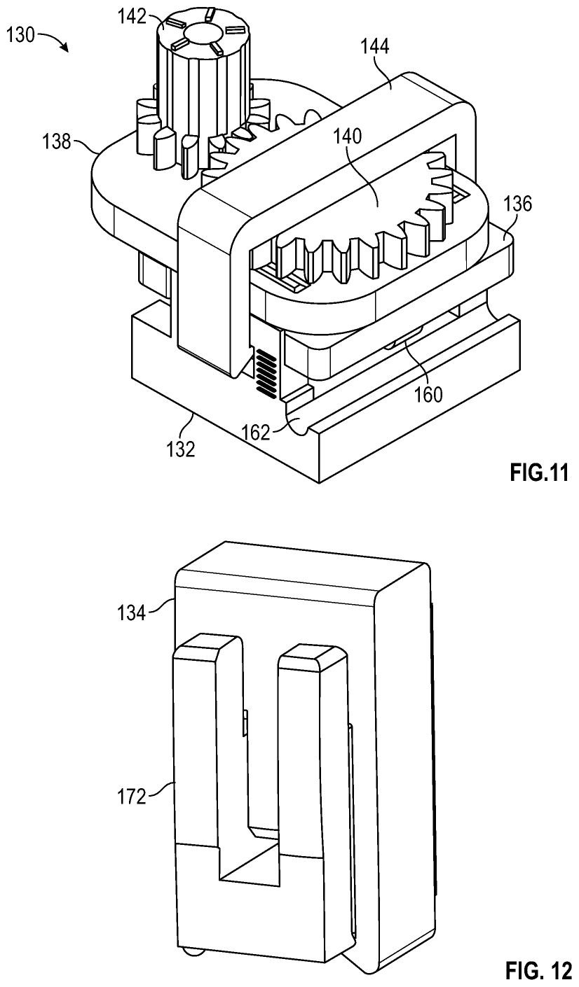

FIG. 11 illustrates a variable flow rate setting device 130 that is similar to the device 30 but does not include an initial rate setter integrated into the same unit. FIG. 12 shows a separate initial rate setter 134 and receiver 172 that can be applied to a flow tube separately from the variable device 130. The device 130 comprises a dial 142 coupled by gears to a screw shaft 140, which is coupled be threads to a compression platform 136 with projection 160 for compressing a tube. The base 132 includes a trough 162 that receives the tube. A bracket 138 attaches to the base and retains the dial 142 and screw shaft 140, while a retainer 144 extends around the top and sides and connects to the base 132 to restrict vertical motion of the screw shaft. The separate receiver 172 can be attachable to the base 132 to can be applied to a tube spaced apart from the base.

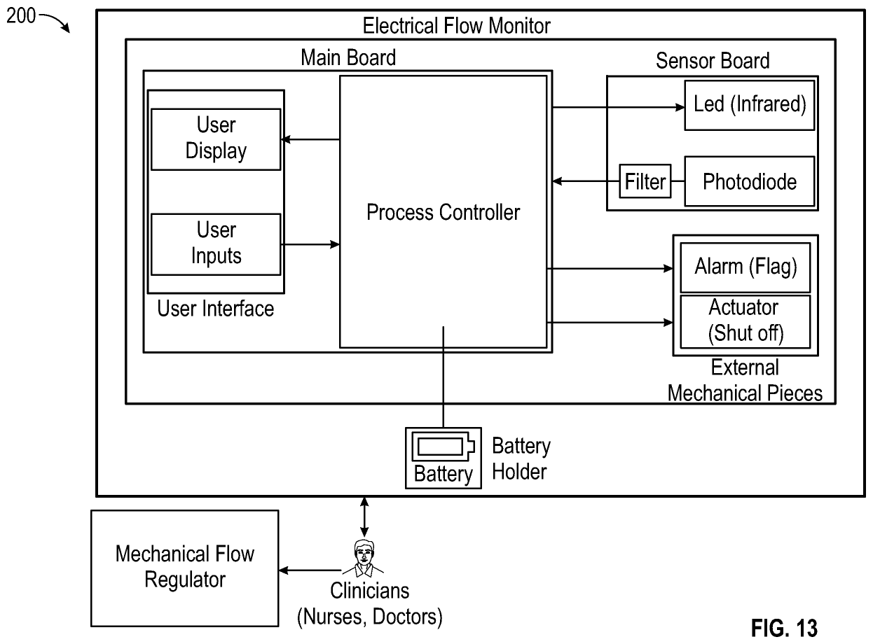

As shown in FIG. 1, the system can include a drip chamber 8 and flow rate monitor 16, a user alert 12, and a shutoff device 14. These components can collectively work together as a monitoring system, an example of which is schematically illustrated in FIG. 13 as monitoring system 200. Exemplary interconnections within the monitoring system 200 are shown in FIG. 13. The system 200 can include a process controller unit, for example a 16-bit microcontroller (MCU), user interface components, sensor board components, an alarm system, a shutoff actuator, a battery, and/or other components. The system 200 can operate at low power to conserve power over a long duration. The system 200 can also be physically couplable to the drip chamber 8, such that the system 200 can monitor the drips within the drip chamber. In addition to the low power and attachability features, the flow rate monitoring system 200 may also alert a clinical staff in case the flow rate deviates by a certain amount and time, such as 5% for 5+ minutes. In some embodiments, alert systems and shut off systems can be one-shot with manual reset, thereby minimizing the continuous power consumption compared to a lighted alert. One AA battery, or other small and low cost battery, can be sufficient to power the monitoring system 200.

The system 200 can also be very compact and lightweight, such as being no larger than 100 mm×76 mm×32 mm, and no heavier than 150 g. The flow rate can be primarily computed based on the drops detected within the drip chamber 8 over a time interval. The system can also include a weight-based sensor that uses change in weight (via accumulation of drops of fluid in the drip chamber) over a time interval to compute the flow rate.

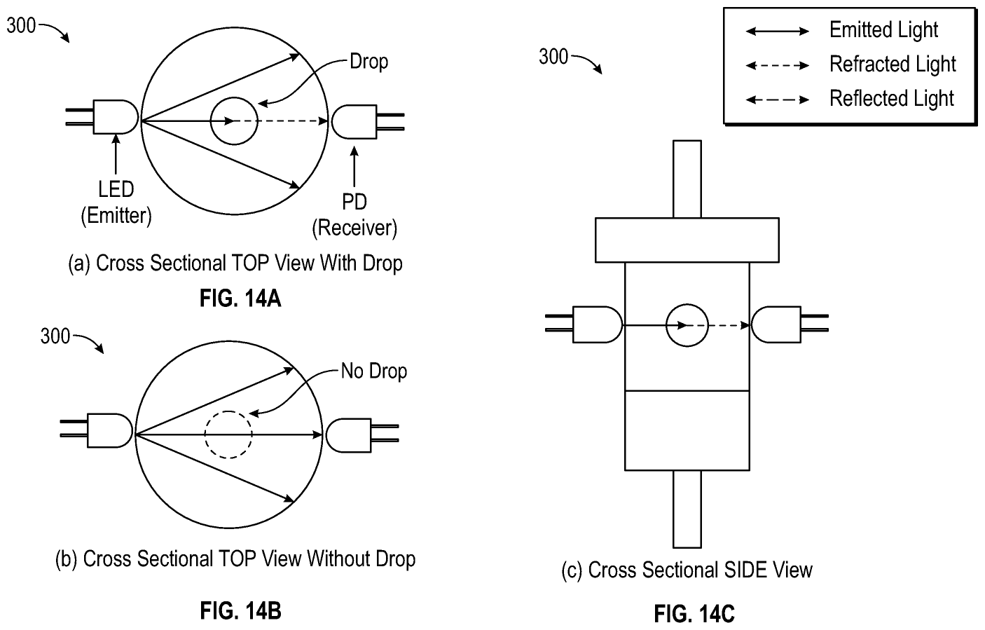

The sensor(s) used to detect drops can comprise, in some examples, light emitting diodes (LED), such as with wavelength in the range of 650-1000 nm (10 m). The emitted light can be collected by a photodiode (PD) or other detectors. Using the photodiode configuration, the detection signal can comprise a negative pulse. An exemplary photodiode configuration 300 is shown in FIGS. 14A-14C. In this example, an LED emits light that crosses the drip chamber and is sensed at an opposite side of the drip chamber by one or more PD. If a drip is present, the light is attenuated by the drip and not sensed (or sensed at a lower level) by the PD. If no drip is present, the PD senses the light at a full level.

The photodiode configuration 300 can function based on how much light the drop absorbs, refracts, and reflects. In some embodiments, the photodiode configuration can detect drops using a negative pulse from the baseline signal. The drop interrupts the beam and reduces the amount of light going to the PD. There can still be a positive signal due to light rays bouncing off the walls and refracting through the drop, but the pulse of an unknown percentage can still be detectable.

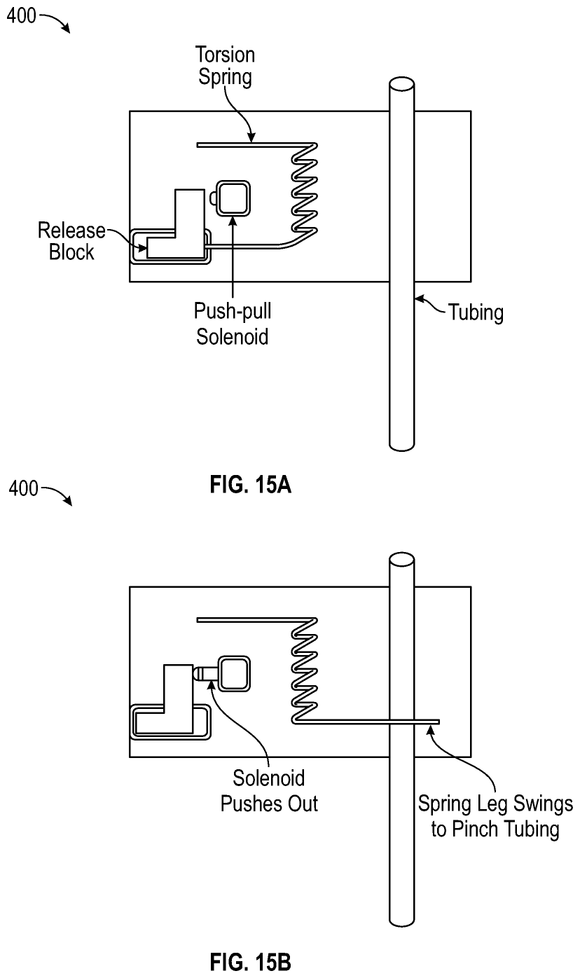

An exemplary emergency shutoff mechanism 400 is shown in FIGS. 15A and 15B. The shutoff mechanism 400 can be analogous to a mouse-trap design. For example, it can be connected to the flow rate monitor and can comprise three main parts: a push-pull solenoid, a release block, and a torsion spring. While the device is operating at a normal flow rate, one of the torsion spring legs can be held back by the release block (FIG. 15A). When the monitor detects a significant deviation from the preset flow rate, the emergency shutoff can trigger, causing the solenoid to push the release block away from the spring leg. The leg can then swing to pinch on the tubing enough so that the IV fluid flow is stopped (FIG. 15B).

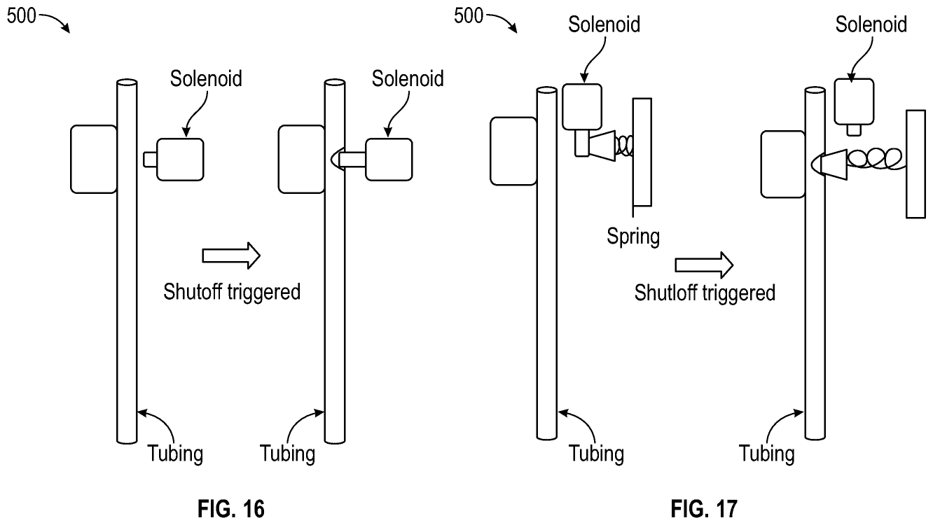

FIG. 16 illustrates a simple shutoff system 500 that uses a solenoid actuator to pinch the tubing. The solenoid pushes out when activated, with the solenoid arm directly pinching the tubing against a rigid surface.

FIG. 17 illustrates another solenoid-based shutoff system 600. Here, the solenoid pulls an arm back when activated to release a spring-biased compressor. When flow is normal and the monitor is performing normally, the solenoid arm blocks compressor, keeping the spring compressed. When the shutoff is activated, the solenoid pulls its arm back, allowing the spring to push the compressor onto the tubing and pinch it closed.

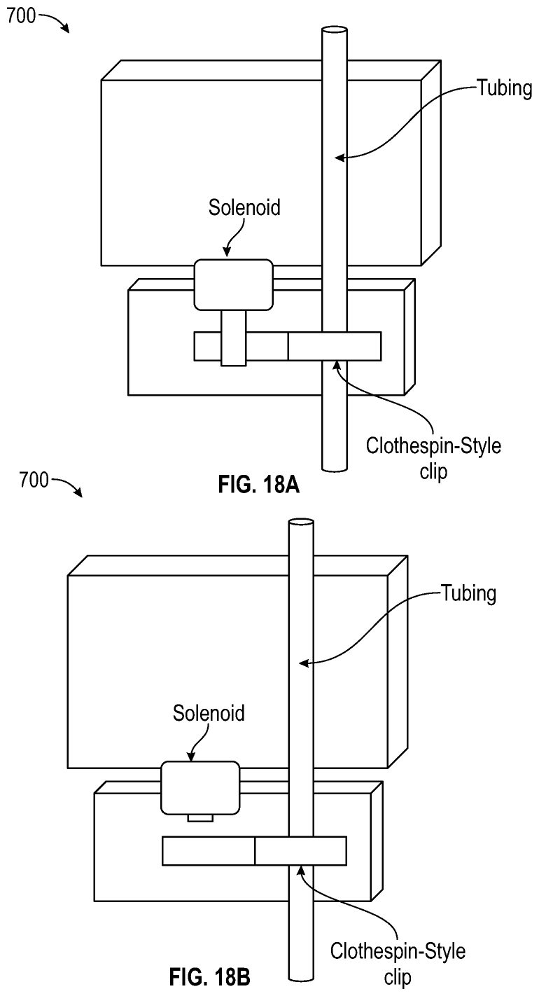

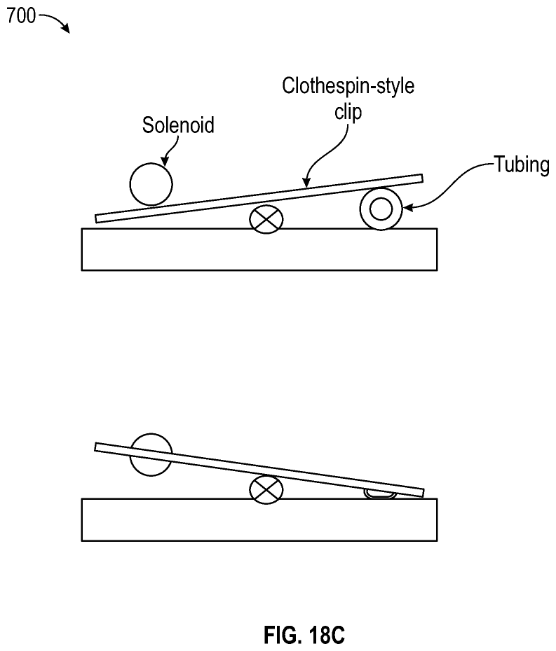

FIGS. 18A-18C illustrate another shutoff system 700 that uses a clothespin-style clip that pinches the tubing closed when the shutoff is activated. When the monitor is operating normally, a solenoid arm holds the clothespin open (FIG. 18A and top of FIG. 18C). As soon as the shutoff is activated, the solenoid arm pulls back (FIG. 18B), setting one end of the clip free and causing the other end of the clip to pinch down on the tubing (bottom of FIG. 18C). The clamping motion can be actuated by a torsion spring that is part of the clothespin-style clip.

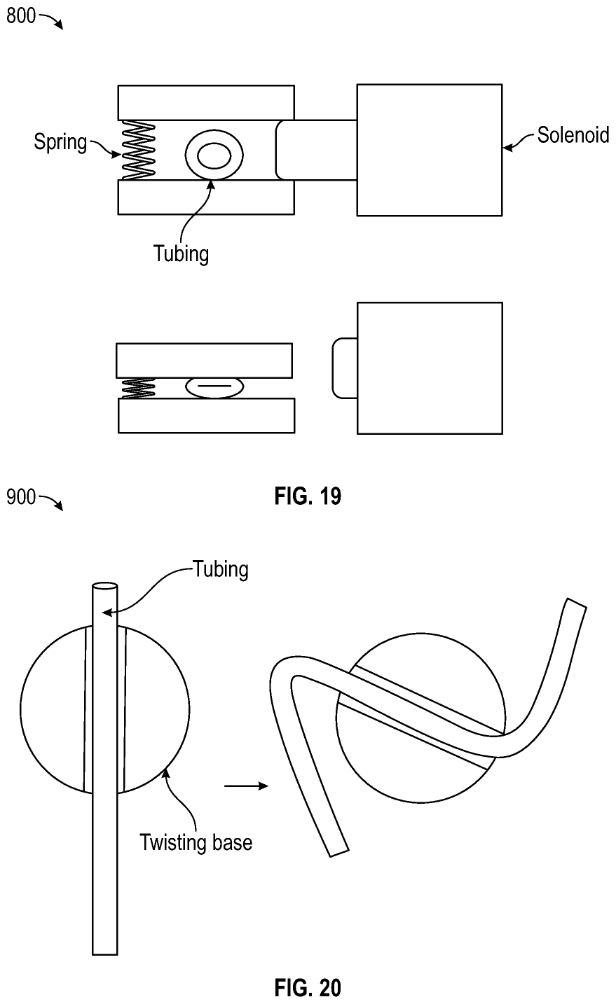

FIG. 19 illustrates a clamp-style shutoff system 800, where a pull-type solenoid arm holds open a clamp, through which the tubing runs (top of FIG. 19). An expanded spring provides tension that will close the clamp onto the tubing when the solenoid arm pulls back at shutoff activation (bottom of FIG. 19).

FIG. 20 illustrates a twisting-type shutoff system 900, where the tubing is held straight within a twisting base during normal operation and flow (left of FIG. 20). A torsion spring provides tension that causes the twisting base to rotate when the shutoff activates, thereby twisting the tubing itself (right side of FIG. 20). This twist can causes two pinches in the tubing where the flow of liquid will be stopped.

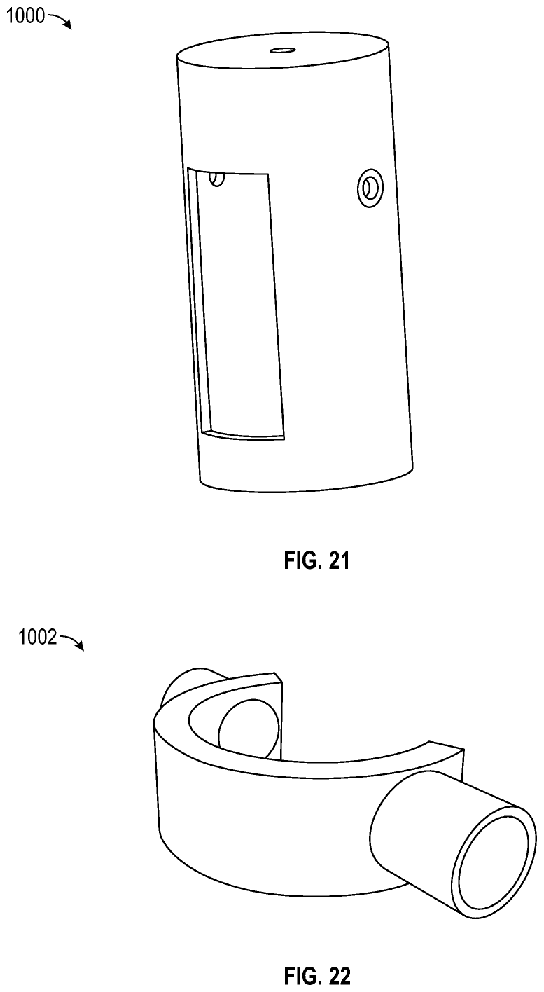

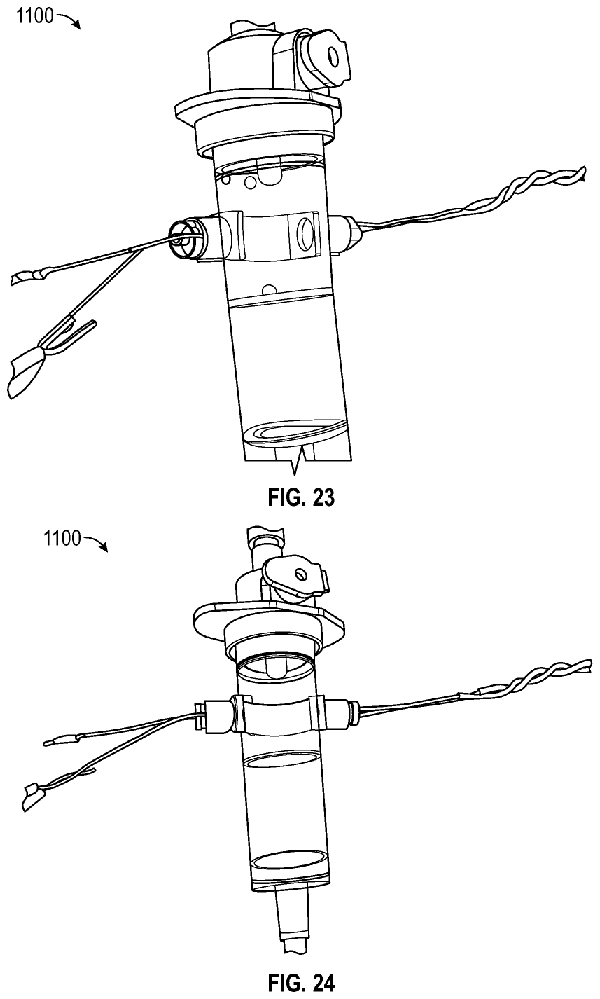

FIG. 21 illustrates an exemplary drip chamber case 1000. FIG. 22 illustrates an exemplary case bracket 1002 that attaches onto the drip chamber case 1000 to hold optical monitoring components. The drip chamber case 1000 that includes a window/opening to such that drips within are visible to user. The case 1000 and/or the bracket 1002 can be 3D printed, for example. FIGS. 23 and 24 show an exemplary drip chamber 1100 that includes the case 1000 and bracket 1002, with other structural elements, along with associated electrical/optical monitoring components coupled to the bracket for monitoring the drips within the chamber.

### Exemplary Hardware and Software Features for Flow Rate Monitoring

Exemplary hardware for the flow rate monitor system can include:
Arduino Uno or Mega microcontrollers
Transimpedance amplifier custom PCB to amplify the signal from the optical sensors, connected to the Arduino pins, powered by the 3.3V pin and the signal is fed into the AO (analog input) pin on the Arduino board
Nokia 5110 display
   Exemplary software can include:
Using a timer interrupt that samples the sensor output every 5 ms (200 Hz)
Drop detection algorithm:
   Calculates the median value of the last 10 samples to filter out noise in real time and create a stable baseline;
   Uses a pre-set threshold value that detects the drop in voltage caused by the drop passing in between the sensors. It is considered a drop when the voltage reading drops below a value of baseline—threshold then returns back up to the baseline;
   Ignores any dips in the signal that might be considered a drop that occur within <40 ms of each other since a physical drop takes around 40 ms to pass through the sensor so it will be physically impossible for that to happen. This was determined by looking at oscilloscope readings of the output signal as the drops pass by the sensors.
Storing the ms between each drop in an array of 10 elements long and we use these 10 values to calculate the average time between drops which is then used to calculate average flow rate.

Flow Rate=3600000/(GTT_FACTOR*average time in ms) Equation:
Upon startup, the code to start flow rate calculation changes AFTER the first drop is detected (so starting from the 2nd drop detected) in order to provide a more accurate reading to the user. This solves the problem of an artificially larger flow rate being displayed at the first drop because of the timing issue of the user not being able to start the monitor up simultaneously with the start of the formation of a new drop.

For purposes of this description, certain aspects, advantages, and novel features of the embodiments of the inventive technology are described herein. The disclosed methods and systems should not be construed as limiting in any way. Instead, the present disclosure is directed toward all novel and nonobvious features and aspects of the various disclosed embodiments, alone and in various combinations and sub-combinations with one another. The methods, apparatuses, and systems are not limited to any specific aspect or feature or combination thereof, nor do the disclosed embodiments require that any one or more specific advantages be present or problems be solved.

Although the operations of some of the disclosed methods are described in a particular, sequential order for convenient presentation, it should be understood that this manner of description encompasses rearrangement, unless a particular ordering is required by specific language. For example, operations described sequentially may in some cases be rearranged or performed concurrently. Moreover, for the sake of simplicity, the specification and attached figures may not show all the various ways in which the disclosed methods can be used in conjunction with other methods.

The singular terms “a”, “an”, and “the” include plural referents unless context clearly indicates otherwise. The term “comprises” means “includes without limitation.” The term “coupled” means physically linked and does not exclude intermediate elements between the coupled elements. The term “and/or” means any one or more of the elements listed. Thus, the term “A and/or B” means “A”, “B” or “A and B.”

In view of the many possible embodiments to which the principles of the disclosed technology may be applied, it should be recognized that the illustrated embodiments are only preferred examples and should not be taken as limiting the scope of the disclosure. Rather, the scope of the disclosure is at least as broad as the following claims.

1. An IV system comprising:
a conduit for conducting an IV fluid from an IV storage container to a patient, the conduit comprising one or more tube portions coupled in series;
a flow rate monitor configured to monitor a flow rate of the IV fluid through the conduit;
a first flow regulator coupled to a first tube portion of the conduit and configured to restrict flow therethrough; and
a second flow regulator coupled to a second tube portion of the conduit and configured to restrict flow therethrough, wherein the first regulator is positioned upstream from the second regulator along the conduit;
wherein a first flow regulator comprises an initial rate setter that compresses the first tube portion a fixed amount to set an initial flow rate through the conduit; and
wherein the second flow regulator comprises a user-adjustable mechanical compression device that compresses the second tube portion to a variable degree to fine-tune the flow rate through the conduit to a precise flow rate that is lower than the initial flow rate.

2. The system of claim 1, wherein the second flow regulator comprises a user-actuatable dial that is mechanically coupled to a compressor platform, where rotational motion of a dial generates linear motion of the compressor platform, and wherein linear motion of the compressor platform compresses the second tube portion to a corresponding degree.

3. The system of claim 2, wherein the second flow regulator further comprises a train of gears coupling the dial to the compressor platform, wherein the train of gears transforms gross rotational motion of the dial to fine linear motion of the compressor platform to enable precise control of the flow rate of the IV fluid to the patient.

4. The system of claim 2, wherein the second flow regulator further comprises a screw shaft having a geared portion that is engaged with a geared portion of the dial, and the screw shaft also has a threaded portion that is engaged with a threaded portion of the compression platform, such that rotation of the dial by the user causes the geared portion of the dial to turn the geared portion of the screw shaft, and such that turning of the geared portion of the screw shaft causes the threaded portion of the screw shaft to turn and to thereby drive the compression platform linearly relative to the second tube portion.

5. The system of claim 1, wherein the first flow regulator and the second flow regulator are two part of a continuous device and share a common base structure, and wherein the first tube portion and the second tube portion are parts of one tube.

6. The system of claim 1, wherein the first and second flow regulators are positioned downstream of the flow rate monitor along the conduit.

7. The system of claim 1, further comprising a shutoff device coupled to the conduit, wherein the shutoff device is operable in a first state in which flow through the conduit is not prevented by the shutoff valve and in a second state in which flow through the conduit is prevented by the shutoff;
wherein the system further comprises a controller operably coupled to the flow rate monitor, wherein the controller is configured to compare a flow rate measured by the flow rate monitor to a predetermined flow rate threshold, and wherein the controller is operable to cause the shutoff to change between the first state and the second state in response to the comparison.

8. The system of claim 7, further comprising an indicator device operably coupled to the controller, wherein the indicator device is configured to present an indication to a user based on the comparison.

9. The system of claim 1, wherein the first flow regulator is configured to restrict flow through the conduit to approximately 20 mL/h to approximately 30 mL/h, and the second flow regulator is operable to restrict flow through the conduit to as low as 1 mL/h.

10. The system of claim 1, wherein the system includes a drip chamber coupled along the conduit and the flow rate monitor includes a light source and an optical sensor coupled to the drip chamber, and wherein the flow rate monitor is configured to detect drips of fluid within the drip chamber to determine a flow rate through the conduit.

11. The system of claim 11, wherein the light source is positioned on a first side of the drip chamber and the optical sensor is positioned on a second side of the drip chamber opposite the first side of the drip chamber.

12. The system of claim 11, wherein the flow rate monitor includes a weight sensor that measures a weight of the fluid in the drip chamber.

13. A flow regulator system for an IV fluid conduit, the system comprising:
a first flow regulator coupled to a first tube portion of the conduit and configured to restrict flow therethrough; and
a second flow regulator coupled to a second tube portion of the conduit and configured to restrict flow therethrough, wherein the first regulator is positioned upstream from the second regulator along the conduit;
wherein the first flow regulator and the second flow regulator are two parts of a contiguous device and share a common base structure, and wherein the first tube portion and the second tube portion are parts of one continuous tube;
wherein a first flow regulator comprises an initial rate setter that compresses the first tube portion a fixed amount to set an initial flow rate through the conduit; and
wherein the second flow regulator comprises a user-actuatable dial that is mechanically coupled to a compressor platform, where rotational motion of a dial generates linear motion of the compressor platform, and wherein linear motion of the compressor platform compresses the second tube portion to a corresponding degree to fine-tune the flow rate through the conduit to a precise flow rate that is lower than the initial flow rate.

14. The system of claim 13, wherein the second flow regulator further comprises a train of gears coupling the dial to the compressor platform, wherein the train of gears transforms gross rotational motion of the dial to fine linear motion of the compressor platform to enable precise control of the flow rate of the IV fluid to the patient.

15. The system of claim 13, wherein the second flow regulator further comprises a screw shaft having a geared portion that is engaged with a geared portion of the dial, and the screw shaft also has a threaded portion that is engaged with a threaded portion of the compression platform, such that rotation of the dial by the user causes the geared portion of the dial to turn the geared portion of the screw shaft, and such that turning of the geared portion of the screw shaft causes the threaded portion of the screw shaft to turn and to thereby drive the compression platform linearly relative to the second tube portion.

16. A method of controlling flow through an IV system, the method comprising:
providing an IV system comprising:
a drip chamber;
an IV fluid bag in fluid communication with an inlet of the drip chamber; and
a tube in fluid communication with an outlet of the drip chamber;
restricting a flow rate of fluid through the tube with a first regulator that sets an initial flow rate;
further restricting the flow rate of fluid through the tube with a second regulator downstream of the first regulator to a target flow rate that is less than the initial flow rate; and
monitoring the flow rate of fluid through drip chamber with a flow rate monitor.

17. The method of claim 16, further comprising:
inputting a predetermined flow rate threshold value into the flow rate monitor; and
comparing flow rate data from the flow rate monitor to the predetermined flow rate threshold value.

18. The method of claim 17, further comprising preventing flow of fluid through the IV system in response to the comparison when the flow rate data exceeds the predetermined flow rate threshold value.

19. The method of claim 17, further comprising adjusting the second regulator in response to the comparison to fine tune the target flow rate.

20. The method of claim 16, wherein monitoring the flow rate of fluid through the drip chamber comprises illuminating at least a portion of the drip chamber with a light source and detecting light from the light source, wherein detecting light from the light source comprises detecting light reflected by or refracted by a drip within the drip chamber.
